(19) United States
(12) Patent Application Publication
(10) Pub. No.: US 2023/0165474 A1
(43) Pub. Date: Jun. 1, 2023

(54) Methods to Simulate Metrics of Vascular Function From Clinical Data

(71) Applicant: Massachusetts Institute of Technology, Cambridge, MA (US)

(72) Inventors: Jonathan Brown, Newton, MA (US); Elazer R. Edelman, Brookline, MA (US); Michael Murphy, Concord, MA (US)

(21) Appl. No.: 17/789,847

(22) PCT Filed: Feb. 25, 2022
(86) PCT No.: PCT/US2022/017877

§ 371 Date: Jun. 29, 2022

Related U.S. Application Data
(60) Provisional application No. 63/154,180, filed on Feb. 26, 2021.

(51) Int. Cl. A61B 5/021; A61B 5/026; A61B 5/00

(52) U.S. Cl. CPC A61B 5/02108; A61B 5/026; A61B 5/7257; A61B 5/05

(57) ABSTRACT

A method includes determining, by a computer processor coupled to memory, data associated with a patient, the data comprising a heart rate value, a peak velocity through aortic valve value, and a stroke volume value; generating a flow waveform for the patient using the data; determining a set of candidate vascular impedance (VI) values for the patient based at least in part on the flow waveform, the set of candidate VI values comprising first and second VI values; determining a first pressure waveform using the flow waveform and the first VI value; determining a second pressure waveform using the flow waveform and the second VI value; determining a blood pressure value for the patient; determining that the first pressure waveform is a closer match to the blood pressure value than the second pressure waveform; and determining that the patient has a vascular impedance of the first VI value.

### CROSS-REFERENCE TO RELATED APPLICATIONS

This application claims priority to U.S. Provisional Application No. 63/154,180, filed Feb. 26, 2021, the content of which is incorporated herein by reference in its entirety.

### BACKGROUND

The present disclosure is generally in the field of medical diagnostics, including methods for assessing vascular impedance in a patient.

Aortic Stenosis (AS) is one of the most common types of cardiovascular disease. Given the aging U.S. population, it is estimated by 2030 that there will be 72 million Americans 65 years and older and approximately 4 million of these individuals will have moderate or severe AS (See Thaden et al., The global burden of aortic stenosis. Prog Cardiovasc Dis 56, 565-571 (2014))). The field of Aortic Valve Replacement (AVR) and the recent introduction of Transcatheter Aortic Valve Replacement (TAVR) are poised to become the dominant method of AVR in the coming years. However, there is still much debate over what are the best metrics to track the progression of AS and determine the correct timing of intervention.

Given the current interventional selection criteria in those who undergo an AVR, it has been found that up to ⅓ of patients derive no benefit (Arnold et al., Predictors of poor outcomes after transcatheter aortic valve replacement: results from the PARTNER (Placement of Aortic Transcatheter Valve) trial. Circulation 129, 2682-2690 (2014)). Given that in AS disease progression is highly variable new metrics for tracking AS progression and determining points for intervention are crucial. Further, the cost of TAVR is significant with the device alone costing ˜$32,000. Thus, there is significant interest in defining when and who are the best candidates for these procedures.

Traditional metrics that dictate treatment normally come from patient symptoms, but objective metrics are all derived from echocardiographic data. Yet even these quantitative echocardiographic metrics are known to be mildly predictive when it comes to procedural success. It has further been shown that the vascular system plays an important role in this disease (See Ben-Assa et al., Ventricular stroke work, and vascular impedance refine the characterization of patients with aortic stenosis. Sci. Transl. Med. (2019); Lindman et al., Blood Pressure and Arterial Load After Transcatheter Aortic Valve Replacement for Aortic Stenosis CLINICAL PERSPECTIVE. Circ. Cardiovasc. Imaging 10, e006308 (2017); Yotti et al., Systemic Vascular Load in Calcific Degenerative Aortic Valve Stenosis. J. Am. Coll. Cardiol. 65, 423-433 (2015)). However, no easily available and routine method exists to measure vascular function.

Vascular impedance is one metric that allows for the complete quantification of vascular function and principally the total afterload imposed by the vascular system. Vascular impedance is expressed as the ratio of the frequency components of pressure and flow waveforms taken at any point within the vascular system.

In practice, invasive blood pressure and flow waveforms are traditionally needed to calculate vascular impedance, but these types of measurements are not practical in all patients on a large scale and for longitudinal tracking. It therefore would be desirable to provide methods and systems for determining vascular impedance using minimal clinical data obtained from routine standard of care visits.

### BRIEF SUMMARY

In one aspect, a method is provided that includes: determining, by one or more computer processors coupled to memory, data associated with a patient, the data comprising a heart rate value, a peak velocity through aortic valve value, and a stroke volume value; generating a flow waveform for the patient using the data; determining a set of candidate vascular impedance values for the patient based at least in part on the flow waveform, the set of candidate vascular impedance values comprising a first vascular impedance value and a second vascular impedance value; determining a first pressure waveform using the flow waveform and the first vascular impedance value; determining a second pressure waveform using the flow waveform and the second vascular impedance value; determining a blood pressure value for the patient; determining that the first pressure waveform is a closer match to the blood pressure value than the second pressure waveform; and determining that the patient has a vascular impedance of the first vascular impedance value.

In another aspect, a method is provided for assessing a patient potentially in need of an aortic valve replacement procedure. The method may include determining, by one or more computer processors coupled to memory, echocardiographic data associated with the patient, the echocardiographic data comprising a heart rate value, a peak velocity through aortic valve value, an aortic valve area value, and a stroke volume value; generating a flow waveform for the patient using the echocardiographic data; determining a set of candidate vascular impedance values for the patient based at least in part on the flow waveform, the set of candidate vascular impedance values comprising a first vascular impedance value and a second vascular impedance value; determining a first pressure waveform using the flow waveform and the first vascular impedance value; determining a second pressure waveform using the flow waveform and the second vascular impedance value; determining a blood pressure value for the patient; determining that the first pressure waveform is a closer match to the blood pressure value than the second pressure waveform; determining that the patient has a vascular impedance of the first vascular impedance value, and then using the vascular impedance to determine whether the patient is a suitable candidate for an aortic valve replacement procedure.

In still another aspect, a device is provided that includes memory configured to store computer-executable instructions; and at least one computer processor configured to access the memory and execute the computer-executable instructions to: determine data associated with a patient, the data comprising a heart rate value, a peak velocity through aortic valve value, and a stroke volume value; generate a flow waveform for the patient using the data; determine a set of candidate vascular impedance values for the patient based at least in part on the flow waveform, the set of candidate vascular impedance values comprising a first vascular impedance value and a second vascular impedance value; determine a blood pressure value for the patient; determine that the first pressure waveform is a closer match to the blood pressure value than the second pressure waveform; and determine that the patient has a vascular impedance of the first vascular impedance value.

### BRIEF DESCRIPTION OF THE DRAWINGS

FIGS. 1A-1B illustrate Fourier Decomposition and corresponding Time Domain Representation, respectively, in accordance with one or more embodiments of the disclosure.

FIG. 2 is an example process flow diagram of the methods, in accordance with one or more embodiments of the disclosure.

FIG. 3 schematically illustrates an example architecture of an electronic device in accordance with one or more embodiments of the disclosure.

The detailed description is set forth with reference to the accompanying drawings. The drawings are provided for purposes of illustration only and merely depict example embodiments of the disclosure. The drawings are provided to facilitate understanding of the disclosure and shall not be deemed to limit the breadth, scope, or applicability of the disclosure. The use of the same reference numerals indicates similar, but not necessarily the same or identical components. Different reference numerals may be used to identify similar components. Various embodiments may utilize elements or components other than those illustrated in the drawings, and some elements and/or components may not be present in various embodiments. The use of singular terminology to describe a component or element may, depending on the context, encompass a plural number of such components or elements and vice versa.

### DETAILED DESCRIPTION

It has been discovered that with the aid of computational modeling combined with minimal clinical data obtained from routine standard of care visits, calculation of vascular impedance is possible.

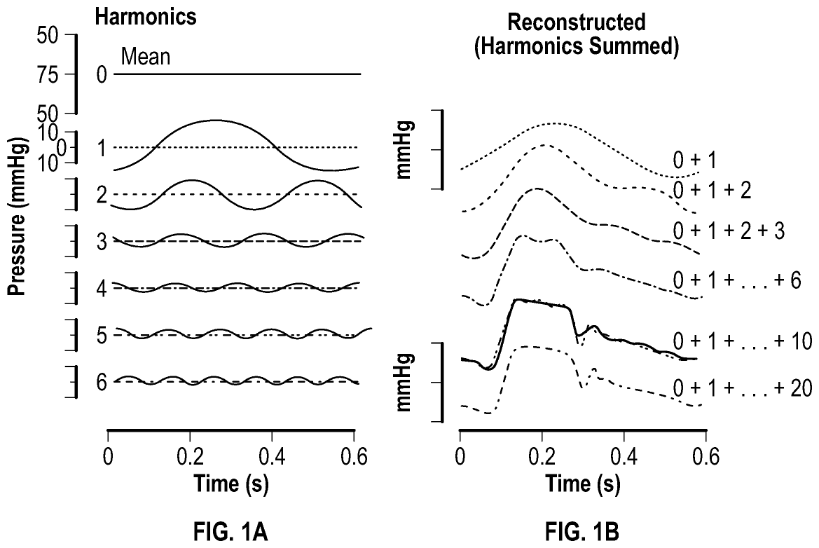

Frequency analysis of both pressure and flow waveforms provides a unique type of analysis that is not traditionally used in clinical medicine. In contrast to examining waveforms as they are captured traditionally, in the time domain, each waveform can be broken down into its component with the use of Fourier decomposition (See Nichols et al., Blood Flow in Arteries Theoretical, Experimental and Clinical Principles, sixth edition. (Cambridge University Press, 2011)) as illustrated in FIGS. 1A-1B. Each one of these components provides a functional description of the vasculature. Principally the higher harmonics representing the characteristic properties of the system, that is stiffness, and the lower harmonics representing the pulsatile components of the system.

Technical Description

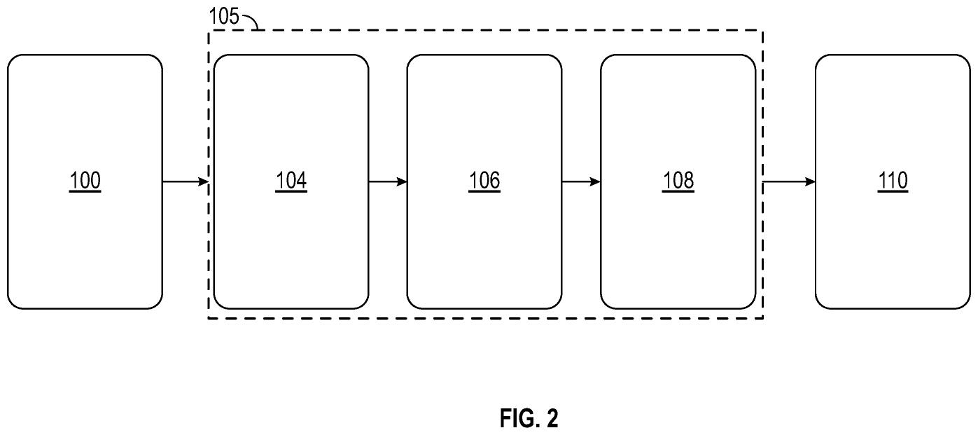

In some embodiments, a method is provided for simulating vascular impedance by generating a patient-specific flow waveform defined by parameters of peak velocity through the aortic valve, stroke volume, heart rate, and aortic valve area. For example, this information may be obtained from a standard echocardiographic evaluation of patients. A set of candidate impedance spectrums are then generated, multiplied by the FFT decomposition of the flow waveform, and a resulting “back-calculation” via the FFT pressure waveform is obtained for all impedance/flow waveform combinations. Pressure measurements are then obtained and used as a means of reducing the number of candidate impedance/pressure waveforms combinations by limiting those generated pressure waveforms that match the patient-specific input pressures. Further knowledge-based domain bounding criteria such as pressure waveform upstroke, ejection duration range, and diastolic decay, are used to eliminated pressure waveforms, leaving only those that meet physiologic criteria, as illustrated in FIG. 2.

In FIG. 2, input variables (measurements) are obtained in step 100, applied in step 104 to define patient specific flow waveform and impedances to be tested, then the Fourier Transform is used to obtain pressure waveforms for all patient specific flow/impedance combinations in step 106, then domain bounding criteria are applied in step 108 to reduce the number of pressure waveforms to those that meet physiological criteria, to produce in step 110 output variables, which is a set of candidate patient specific impedance values and passing pressure waveforms. Box 105 indicates the steps are repeated for all impedance/flow combinations.

In one aspect, a method is provided that includes: (i) determining, by one or more computer processors coupled to memory, data associated with a patient, the data comprising a heart rate value, a peak velocity through aortic valve value, and a stroke volume value, and optionally an aortic valve area value; (ii) generating a flow waveform for the patient using the data; (iii) determining a set of candidate vascular impedance values for the patient based at least in part on the flow waveform, the set of candidate vascular impedance values comprising a first vascular impedance value and a second vascular impedance value; (iv) determining a first pressure waveform using the flow waveform and the first vascular impedance value; (v) determining a second pressure waveform using the flow waveform and the second vascular impedance value; (vi) determining a blood pressure value for the patient; (vii) determining that the first pressure waveform is a closer match to the blood pressure value than the second pressure waveform; and (ix) determining that the patient has a vascular impedance of the first vascular impedance value. In a preferred embodiment, the step of determining the first pressure waveform includes determining the first pressure waveform based at least in part on a Fourier Transform of the flow waveform and the first vascular impedance value.

In the presently disclosed methods, the peak velocity through the aortic valve, stroke volume, heart rate, and aortic valve area include the component parts of each of these inputs. For example, stroke volume is a routinely calculated metric, and the presently disclosed methods can be executed with either the component measurements/data used to calculate stroke volume or the stroke volume itself. Accordingly, a claim step reciting, for example, a stroke volume value is met by component part values from which stroke volume can be calculated.

The data associated with the patient may be obtained by echocardiography, MRI, or a combination thereof. The blood pressure value for the patient may be obtained by any conventional means. In some embodiments, the blood pressure is brachial blood pressure.

In some embodiments, the method further includes filtering out the second pressure waveform based at least in part on one or more domain bounding criteria. The one or more domain bounding criteria may include pressure waveform upstroke (positive values thereof), ejection duration range, and diastolic decay.

In some embodiments, the method further includes generating a recommendation for an aortic valve replacement procedure for the patient based at least in part on the vascular impedance. For example, the recommendation may be that the patient should or should not undergo an aortic valve replacement procedure at this time.

In some embodiments, the method further includes generating a predicted outcome of an aortic valve replacement procedure for the patient based at least in part on the vascular impedance. The generating the predicted outcome may include selecting the predicted outcome from a predefined set of outcomes.

In another aspect, a device is provided which includes (i) a memory configured to store computer-executable instructions; and (ii) at least one computer processor configured to access the memory and execute the computer-executable instructions to carry out the steps (i)-(ix) identified above.

Advantages and Improvements Over Existing Methods, Devices, or Materials

While other non-invasive methods of vascular impedance calculation do exist, they rely on expensive equipment and non-standard software methods. Further to use these methods additional time, training, and effort are required. The advantage of the method disclosed herein is that no additional data needs to be collected outside of routine clinical care. This allows broad applicability and no additional training for point of care use. Finally, the computational nature of methods ensures repeatability whereas the other non-invasive methods introduce a degree of inter-operator variability.

Uses and Commercial Applications of the Methods

The presently disclosed methods have commercialization potential, either as a system to augment current echocardiographic devices that are used to track disease progression and points for interventional care, or as a stand-alone system in which clinicians evaluating patient disease state for AVR can use the method described to determine vascular function and ultimately better determine the timing of intervention or predict procedural outcomes. This can help optimize patient care and reduce overall costs.

Devices for Implementing the Method

One or more operations of the methods of FIG. 2 may be described as being performed by a user device, or more specifically, by one or more program module(s), applications, or the like executing on a device. In some embodiments, the device may be a stand-alone computing device and/or integrated into, or operatively in communication with, conventional medical equipment, such as an echocardiograph (ECG) machine or magnetic resonance imaging (MRI) machine, and manual or digital blood pressure measurement monitors or sphygmomanometers.

It should be appreciated, however, that any of the operations of the methods of FIG. 2 may be performed, at least in part, in a distributed manner by one or more other devices, or more specifically, by one or more program module(s), applications, or the like executing on such devices. In addition, it should be appreciated that the processing performed in response to the execution of computer-executable instructions provided as part of an application, program module, or the like may be interchangeably described herein as being performed by the application or the program module itself or by a device on which the application, program module, or the like is executing. While the operations of the methods of FIG. 2 may be described in the context of conventional devices, it should be appreciated that such operations may be implemented in connection with numerous other device configurations.

The operations described and depicted in the illustrative methods depicted in FIG. 2 may be carried out or performed in any suitable order. Additionally, in certain example embodiments, at least a portion of the operations may be carried out in parallel. Furthermore, in certain example embodiments, less, more, or different operations than those depicted in FIG. 2 may be performed.

A system may be provide that is configured to determine the vascular impedance of a patient according to the methods described herein. The system may include an echocardiography ultrasound machine, as known in the art.

Although specific embodiments of the disclosure have been described, one of ordinary skill in the art will recognize that numerous other modifications and alternative embodiments are within the scope of the disclosure. For example, any of the functionality and/or processing capabilities described with respect to a particular device or component may be performed by any other device or component. Further, while various illustrative implementations and architectures have been described in accordance with embodiments of the disclosure, one of ordinary skill in the art will appreciate that numerous other modifications to the illustrative implementations and architectures described herein are also within the scope of this disclosure.

Certain aspects of the disclosure are described above with reference to block and flow diagrams of systems, methods, apparatuses, and/or computer program products according to example embodiments. It will be understood that one or more blocks of the block diagrams and flow diagrams, and combinations of blocks in the block diagrams and the flow diagrams, respectively, may be implemented by execution of computer-executable program instructions. Likewise, some blocks of the block diagrams and flow diagrams may not necessarily need to be performed in the order presented, or may not necessarily need to be performed at all, according to some embodiments. Further, additional components and/or operations beyond those depicted in blocks of the block and/or flow diagrams may be present in certain embodiments.

Accordingly, blocks of the block diagrams and flow diagrams support combinations of means for performing the specified functions, combinations of elements or steps for performing the specified functions, and program instruction means for performing the specified functions. It will also be understood that each block of the block diagrams and flow diagrams, and combinations of blocks in the block diagrams and flow diagrams, may be implemented by special-purpose, hardware-based computer systems that perform the specified functions, elements or steps, or combinations of special-purpose hardware and computer instructions.

Illustrative Device Architecture

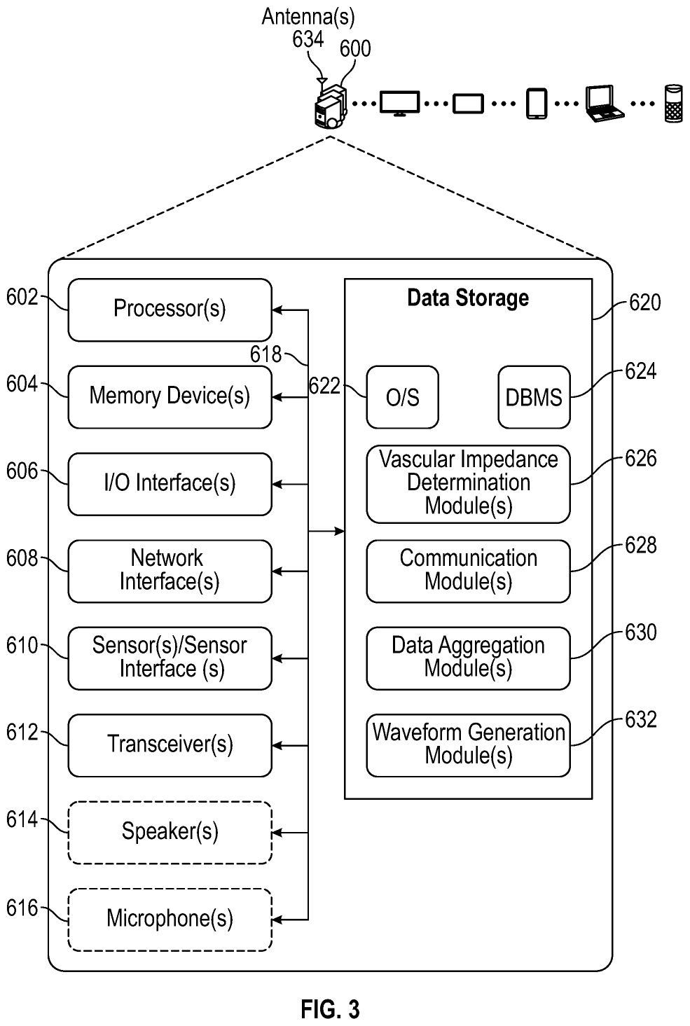

FIG. 3 is a schematic block diagram of an illustrative remote server 600 in accordance with one or more embodiments of the disclosure. The remote server 600 may include any suitable computing device capable of receiving and/or sending data including, but not limited to, a mobile device such as a smartphone, tablet, e-reader, wearable device, or the like; a desktop computer; a laptop computer; or the like.

The remote server 600 may be configured to communicate via one or more networks with one or more servers, search engines, user devices, or the like.

Example network(s) may include, but are not limited to, any one or more different types of communications networks such as, for example, cable networks, public networks (e.g., the Internet), private networks (e.g., frame-relay networks), wireless networks, cellular networks, telephone networks (e.g., a public switched telephone network), or any other suitable private or public packet-switched or circuit-switched networks. Further, such network(s) may have any suitable communication range associated therewith and may include, for example, global networks (e.g., the Internet), metropolitan area networks (MANs), wide area networks (WANs), local area networks (LANs), or personal area networks (PANs). In addition, such network(s) may include communication links and associated networking devices (e.g., link-layer switches, routers, etc.) for transmitting network traffic over any suitable type of medium including, but not limited to, coaxial cable, twisted-pair wire (e.g., twisted-pair copper wire), optical fiber, a hybrid fiber-coaxial (HFC) medium, a microwave medium, a radio frequency communication medium, a satellite communication medium, or any combination thereof.

In an illustrative configuration, the remote server 600 may include one or more processors (processor(s)) 602, one or more memory devices 604 (generically referred to herein as memory 604), one or more input/output (I/O) interface(s) 606, one or more network interface(s) 608, one or more sensors or sensor interface(s) 610, one or more transceivers 612, one or more optional speakers 614, one or more optional microphones 616, and data storage 620. The remote server 600 may further include one or more buses 618 that functionally couple various components of the remote server 600. The remote server 600 may further include one or more antenna(s) 634 that may include, without limitation, a cellular antenna for transmitting or receiving signals to/from a cellular network infrastructure, an antenna for transmitting or receiving Wi-Fi signals to/from an access point (AP), a Global Navigation Satellite System (GNSS) antenna for receiving GNSS signals from a GNSS satellite, a Bluetooth antenna for transmitting or receiving Bluetooth signals, a Near Field Communication (NFC) antenna for transmitting or receiving NFC signals, and so forth. These various components will be described in more detail hereinafter.

The bus(es) 618 may include at least one of a system bus, a memory bus, an address bus, or a message bus, and may permit exchange of information (e.g., data (including computer-executable code), signaling, etc.) between various components of the remote server 600. The bus(es) 618 may include, without limitation, a memory bus or a memory controller, a peripheral bus, an accelerated graphics port, and so forth. The bus(es) 618 may be associated with any suitable bus architecture including, without limitation, an Industry Standard Architecture (ISA), a Micro Channel Architecture (MCA), an Enhanced ISA (EISA), a Video Electronics Standards Association (VESA) architecture, an Accelerated Graphics Port (AGP) architecture, a Peripheral Component Interconnects (PCI) architecture, a PCI-Express architecture, a Personal Computer Memory Card International Association (PCMCIA) architecture, a Universal Serial Bus (USB) architecture, and so forth.

The memory 604 of the remote server 600 may include volatile memory (memory that maintains its state when supplied with power) such as random access memory (RAM) and/or non-volatile memory (memory that maintains its state even when not supplied with power) such as read-only memory (ROM), flash memory, ferroelectric RAM (FRAM), and so forth. Persistent data storage, as that term is used herein, may include non-volatile memory. In certain example embodiments, volatile memory may enable faster read/write access than non-volatile memory. However, in certain other example embodiments, certain types of non-volatile memory (e.g., FRAM) may enable faster read/write access than certain types of volatile memory.

In various implementations, the memory 604 may include multiple different types of memory such as various types of static random access memory (SRAM), various types of dynamic random access memory (DRAM), various types of unalterable ROM, and/or writeable variants of ROM such as electrically erasable programmable read-only memory (EEPROM), flash memory, and so forth. The memory 604 may include main memory as well as various forms of cache memory such as instruction cache(s), data cache(s), translation lookaside buffer(s) (TLBs), and so forth. Further, cache memory such as a data cache may be a multi-level cache organized as a hierarchy of one or more cache levels (L1, L2, etc.).

The data storage 620 may include removable storage and/or non-removable storage including, but not limited to, magnetic storage, optical disk storage, and/or tape storage. The data storage 620 may provide non-volatile storage of computer-executable instructions and other data. The memory 604 and the data storage 620, removable and/or non-removable, are examples of computer-readable storage media (CRSM) as that term is used herein.

The data storage 620 may store computer-executable code, instructions, or the like that may be loadable into the memory 604 and executable by the processor(s) 602 to cause the processor(s) 602 to perform or initiate various operations. The data storage 620 may additionally store data that may be copied to memory 604 for use by the processor(s) 602 during the execution of the computer-executable instructions. Moreover, output data generated as a result of execution of the computer-executable instructions by the processor(s) 602 may be stored initially in memory 604, and may ultimately be copied to data storage 620 for non-volatile storage.

More specifically, the data storage 620 may store one or more operating systems (O/S) 622; one or more database management systems (DBMS) 624; and one or more program module(s), applications, engines, computer-executable code, scripts, or the like such as, for example, one or more vascular impedance determination module(s) 626, one or more communication module(s) 628, one or more (cardiac) data aggregation module(s) 630, and/or one or more waveform generation module(s) 632. Some or all of these module(s) may be sub-module(s). Any of the components depicted as being stored in data storage 620 may include any combination of software, firmware, and/or hardware. The software and/or firmware may include computer-executable code, instructions, or the like that may be loaded into the memory 604 for execution by one or more of the processor(s) 602. Any of the components depicted as being stored in data storage 620 may support functionality described in reference to correspondingly named components earlier in this disclosure.

The data storage 620 may further store various types of data utilized by components of the remote server 600. Any data stored in the data storage 620 may be loaded into the memory 604 for use by the processor(s) 602 in executing computer-executable code. In addition, any data depicted as being stored in the data storage 620 may potentially be stored in one or more datastore(s) and may be accessed via the DBMS 624 and loaded in the memory 604 for use by the processor(s) 602 in executing computer-executable code. The datastore(s) may include, but are not limited to, databases (e.g., relational, object-oriented, etc.), file systems, flat files, distributed datastores in which data is stored on more than one node of a computer network, peer-to-peer network datastores, or the like. In FIG. 3, the datastore(s) may include, for example, user preference information, historical vascular impedance information, and other information.

The processor(s) 602 may be configured to access the memory 604 and execute computer-executable instructions loaded therein. For example, the processor(s) 602 may be configured to execute computer-executable instructions of the various program module(s), applications, engines, or the like of the remote server 600 to cause or facilitate various operations to be performed in accordance with one or more embodiments of the disclosure. The processor(s) 602 may include any suitable processing unit capable of accepting data as input, processing the input data in accordance with stored computer-executable instructions, and generating output data. The processor(s) 602 may include any type of suitable processing unit including, but not limited to, a central processing unit, a microprocessor, a Reduced Instruction Set Computer (RISC) microprocessor, a Complex Instruction Set Computer (CISC) microprocessor, a microcontroller, an Application Specific Integrated Circuit (ASIC), a Field-Programmable Gate Array (FPGA), a System-on-a-Chip (SoC), a digital signal processor (DSP), and so forth. Further, the processor(s) 602 may have any suitable microarchitecture design that includes any number of constituent components such as, for example, registers, multiplexers, arithmetic logic units, cache controllers for controlling read/write operations to cache memory, branch predictors, or the like. The microarchitecture design of the processor(s) 602 may be capable of supporting any of a variety of instruction sets.

Referring now to functionality supported by the various program module(s) depicted in FIG. 3, the vascular impedance determination module 626 may include computer-executable instructions, code, or the like that responsive to execution by one or more of the processor(s) 602 may perform functions including, but not limited to, determining a set of candidate vascular impedance values for the patient based at least in part on the flow waveform, the set of candidate vascular impedance values comprising a first vascular impedance value and a second vascular impedance value, determining a first pressure waveform using the flow waveform and the first vascular impedance value; determining a second pressure waveform using the flow waveform and the second vascular impedance value; applying domain bounding criteria to reduce the number of pressure waveforms to those that meet physiologic criteria; obtaining a set of candidate patient specific impedance values and passing pressure waveforms, and the like.

The communication module(s) 628 may include computer-executable instructions, code, or the like that responsive to execution by one or more of the processor(s) 602 may perform functions including, but not limited to, communicating with one or more devices, for example, via wired or wireless communication, communicating with remote servers, communicating with remote datastores, sending or receiving notifications or commands/directives, communicating with cache memory data, communicating with user devices, and the like.

The data aggregation module(s) 630 may include computer-executable instructions, code, or the like that responsive to execution by one or more of the processor(s) 602 may perform functions including, but not limited to, receiving and analyzing blood pressure values of a patient; receiving and analyzing cardiac information of the patient including heart rate, peak velocity through valve, stroke volume, and aortic valve area; and determining ECG and/or MRI data associated with the patient; determining a blood pressure value for the patient, and the like.

The waveform generation module(s) 632 may include computer-executable instructions, code, or the like that responsive to execution by one or more of the processor(s) 602 may perform functions including, but not limited to, defining patient specific flow waveform and impedances to be tested, using the Fourier Transform to obtain pressure waveforms for all patient specific flow/impedance combinations; generating a flow waveform for the patient using the data, and the like.

Referring now to other illustrative components depicted as being stored in the data storage 620, the O/S 622 may be loaded from the data storage 620 into the memory 604 and may provide an interface between other application software executing on the remote server 600 and hardware resources of the remote server 600. More specifically, the O/S 622 may include a set of computer-executable instructions for managing hardware resources of the remote server 600 and for providing common services to other application programs (e.g., managing memory allocation among various application programs). In certain example embodiments, the O/S 622 may control execution of the other program module(s) to for content rendering. The O/S 622 may include any operating system now known or which may be developed in the future including, but not limited to, any server operating system, any mainframe operating system, or any other proprietary or non-proprietary operating system.

The DBMS 624 may be loaded into the memory 604 and may support functionality for accessing, retrieving, storing, and/or manipulating data stored in the memory 604 and/or data stored in the data storage 620. The DBMS 624 may use any of a variety of database models (e.g., relational model, object model, etc.) and may support any of a variety of query languages. The DBMS 624 may access data represented in one or more data schemas and stored in any suitable data repository including, but not limited to, databases (e.g., relational, object-oriented, etc.), file systems, flat files, distributed datastores in which data is stored on more than one node of a computer network, peer-to-peer network datastores, or the like. In those example embodiments in which the remote server 600 is a mobile device, the DBMS 624 may be any suitable light-weight DBMS optimized for performance on a mobile device.

Referring now to other illustrative components of the remote server 600, the input/output (I/O) interface(s) 606 may facilitate the receipt of input information by the remote server 600 from one or more I/O devices as well as the output of information from the remote server 600 to the one or more I/O devices. The I/O devices may include any of a variety of components such as a display or display screen having a touch surface or touchscreen; an audio output device for producing sound, such as a speaker; an audio capture device, such as a microphone; an image and/or video capture device, such as a camera; a haptic unit; and so forth. Any of these components may be integrated into the remote server 600 or may be separate. The I/O devices may further include, for example, any number of peripheral devices such as data storage devices, printing devices, and so forth.

The I/O interface(s) 606 may also include an interface for an external peripheral device connection such as universal serial bus (USB), FireWire, Thunderbolt, Ethernet port or other connection protocol that may connect to one or more networks. The I/O interface(s) 606 may also include a connection to one or more of the antenna(s) 634 to connect to one or more networks via a wireless local area network (WLAN) (such as Wi-Fi) radio, Bluetooth, ZigBee, and/or a wireless network radio, such as a radio capable of communication with a wireless communication network such as a Long Term Evolution (LTE) network, WiMAX network, 3G network, ZigBee network, etc.

The remote server 600 may further include one or more network interface(s) 608 via which the remote server 600 may communicate with any of a variety of other systems, platforms, networks, devices, and so forth. The network interface(s) 608 may enable communication, for example, with one or more wireless routers, one or more host servers, one or more web servers, and the like via one or more of networks.

The antenna(s) 634 may include any suitable type of antenna depending, for example, on the communications protocols used to transmit or receive signals via the antenna(s) 634. Non-limiting examples of suitable antennas may include directional antennas, non-directional antennas, dipole antennas, folded dipole antennas, patch antennas, multiple-input multiple-output (MIMO) antennas, or the like. The antenna(s) 634 may be communicatively coupled to one or more transceivers 612 or radio components to which or from which signals may be transmitted or received.

As previously described, the antenna(s) 634 may include a cellular antenna configured to transmit or receive signals in accordance with established standards and protocols, such as Global System for Mobile Communications (GSM), 3G standards (e.g., Universal Mobile Telecommunications System (UMTS), Wideband Code Division Multiple Access (W-CDMA), CDMA2000, etc.), 4G standards (e.g., Long-Term Evolution (LTE), WiMax, etc.), direct satellite communications, or the like.

The antenna(s) 634 may additionally, or alternatively, include a Wi-Fi antenna configured to transmit or receive signals in accordance with established standards and protocols, such as the IEEE 802.11 family of standards, including via 2.4 GHz channels (e.g., 802.11b, 802.11g, 802.11n), 5 GHz channels (e.g., 802.11n, 802.11ac), or 60 GHz channels (e.g., 802.11ad). In alternative example embodiments, the antenna(s) 634 may be configured to transmit or receive radio frequency signals within any suitable frequency range forming part of the unlicensed portion of the radio spectrum.

The antenna(s) 634 may additionally, or alternatively, include a GNSS antenna configured to receive GNSS signals from three or more GNSS satellites carrying time-position information to triangulate a position therefrom. Such a GNSS antenna may be configured to receive GNSS signals from any current or planned GNSS such as, for example, the Global Positioning System (GPS), the GLONASS System, the Compass Navigation System, the Galileo System, or the Indian Regional Navigational System.

The transceiver(s) 612 may include any suitable radio component(s) for—in cooperation with the antenna(s) 634—transmitting or receiving radio frequency (RF) signals in the bandwidth and/or channels corresponding to the communications protocols utilized by the remote server 600 to communicate with other devices. The transceiver(s) 612 may include hardware, software, and/or firmware for modulating, transmitting, or receiving—potentially in cooperation with any of antenna(s) 634—communications signals according to any of the communications protocols discussed above including, but not limited to, one or more Wi-Fi and/or Wi-Fi direct protocols, as standardized by the IEEE 802.11 standards, one or more non-Wi-Fi protocols, or one or more cellular communications protocols or standards. The transceiver(s) 612 may further include hardware, firmware, or software for receiving GNSS signals. The transceiver(s) 612 may include any known receiver and baseband suitable for communicating via the communications protocols utilized by the remote server 600. The transceiver(s) 612 may further include a low noise amplifier (LNA), additional signal amplifiers, an analog-to-digital (A/D) converter, one or more buffers, a digital baseband, or the like.

The sensor(s)/sensor interface(s) 610 may include or may be capable of interfacing with any suitable type of sensing device such as, for example, inertial sensors, force sensors, thermal sensors, blood pressure sensors, and so forth. Example types of inertial sensors may include accelerometers (e.g., MEMS-based accelerometers), gyroscopes, and so forth.

The optional speaker(s) 614 may be any device configured to generate audible sound. The optional microphone(s) 616 may be any device configured to receive analog sound input or voice data.

It should be appreciated that the program module(s), applications, computer-executable instructions, code, or the like depicted in FIG. 3 as being stored in the data storage 620 are merely illustrative and not exhaustive and that processing described as being supported by any particular module may alternatively be distributed across multiple module(s) or performed by a different module. In addition, various program module(s), script(s), plug-in(s), Application Programming Interface(s) (API(s)), or any other suitable computer-executable code hosted locally on the remote server 600, and/or hosted on other computing device(s) accessible via one or more networks, may be provided to support functionality provided by the program module(s), applications, or computer-executable code depicted in FIG. 3 and/or additional or alternate functionality. Further, functionality may be modularized differently such that processing described as being supported collectively by the collection of program module(s) depicted in FIG. 3 may be performed by a fewer or greater number of module(s), or functionality described as being supported by any particular module may be supported, at least in part, by another module. In addition, program module(s) that support the functionality described herein may form part of one or more applications executable across any number of systems or devices in accordance with any suitable computing model such as, for example, a client-server model, a peer-to-peer model, and so forth. In addition, any of the functionality described as being supported by any of the program module(s) depicted in FIG. 3 may be implemented, at least partially, in hardware and/or firmware across any number of devices.

It should further be appreciated that the remote server 600 may include alternate and/or additional hardware, software, or firmware components beyond those described or depicted without departing from the scope of the disclosure. More particularly, it should be appreciated that software, firmware, or hardware components depicted as forming part of the remote server 600 are merely illustrative and that some components may not be present or additional components may be provided in various embodiments. While various illustrative program module(s) have been depicted and described as software module(s) stored in data storage 620, it should be appreciated that functionality described as being supported by the program module(s) may be enabled by any combination of hardware, software, and/or firmware. It should further be appreciated that each of the above-mentioned module(s) may, in various embodiments, represent a logical partitioning of supported functionality. This logical partitioning is depicted for ease of explanation of the functionality and may not be representative of the structure of software, hardware, and/or firmware for implementing the functionality. Accordingly, it should be appreciated that functionality described as being provided by a particular module may, in various embodiments, be provided at least in part by one or more other module(s). Further, one or more depicted module(s) may not be present in certain embodiments, while in other embodiments, additional module(s) not depicted may be present and may support at least a portion of the described functionality and/or additional functionality. Moreover, while certain module(s) may be depicted and described as sub-module(s) of another module, in certain embodiments, such module(s) may be provided as independent module(s) or as sub-module(s) of other module(s).

Program module(s), applications, or the like disclosed herein may include one or more software components including, for example, software objects, methods, data structures, or the like. Each such software component may include computer-executable instructions that, responsive to execution, cause at least a portion of the functionality described herein (e.g., one or more operations of the illustrative methods described herein) to be performed.

A software component may be coded in any of a variety of programming languages. An illustrative programming language may be a lower-level programming language such as an assembly language associated with a particular hardware architecture and/or operating system platform. A software component comprising assembly language instructions may require conversion into executable machine code by an assembler prior to execution by the hardware architecture and/or platform.

Another example programming language may be a higher-level programming language that may be portable across multiple architectures. A software component comprising higher-level programming language instructions may require conversion to an intermediate representation by an interpreter or a compiler prior to execution.

Other examples of programming languages include, but are not limited to, a macro language, a shell or command language, a job control language, a script language, a database query or search language, or a report writing language. In one or more example embodiments, a software component comprising instructions in one of the foregoing examples of programming languages may be executed directly by an operating system or other software component without having to be first transformed into another form.

A software component may be stored as a file or other data storage construct. Software components of a similar type or functionally related may be stored together such as, for example, in a particular directory, folder, or library. Software components may be static (e.g., pre-established or fixed) or dynamic (e.g., created or modified at the time of execution).

Software components may invoke or be invoked by other software components through any of a wide variety of mechanisms. Invoked or invoking software components may comprise other custom-developed application software, operating system functionality (e.g., device drivers, data storage (e.g., file management) routines, other common routines and services, etc.), or third-party software components (e.g., middleware, encryption, or other security software, database management software, file transfer or other network communication software, mathematical or statistical software, image processing software, and format translation software).

Software components associated with a particular solution or system may reside and be executed on a single platform or may be distributed across multiple platforms. The multiple platforms may be associated with more than one hardware vendor, underlying chip technology, or operating system. Furthermore, software components associated with a particular solution or system may be initially written in one or more programming languages, but may invoke software components written in another programming language.

Computer-executable program instructions may be loaded onto a special-purpose computer or other particular machine, a processor, or other programmable data processing apparatus to produce a particular machine, such that execution of the instructions on the computer, processor, or other programmable data processing apparatus causes one or more functions or operations specified in the flow diagrams to be performed. These computer program instructions may also be stored in a computer-readable storage medium (CRSM) that upon execution may direct a computer or other programmable data processing apparatus to function in a particular manner, such that the instructions stored in the computer-readable storage medium produce an article of manufacture including instruction means that implement one or more functions or operations specified in the flow diagrams. The computer program instructions may also be loaded onto a computer or other programmable data processing apparatus to cause a series of operational elements or steps to be performed on the computer or other programmable apparatus to produce a computer-implemented process.

Additional types of CRSM that may be present in any of the devices described herein may include, but are not limited to, programmable random access memory (PRAM), SRAM, DRAM, RAM, ROM, electrically erasable programmable read-only memory (EEPROM), flash memory or other memory technology, compact disc read-only memory (CD-ROM), digital versatile disc (DVD) or other optical storage, magnetic cassettes, magnetic tape, magnetic disk storage or other magnetic storage devices, or any other medium which can be used to store the information and which can be accessed. Combinations of any of the above are also included within the scope of CRSM. Alternatively, computer-readable communication media (CRCM) may include computer-readable instructions, program module(s), or other data transmitted within a data signal, such as a carrier wave, or other transmission. However, as used herein, CRSM does not include CRCM.

Although embodiments have been described in language specific to structural features and/or methodological acts, it is to be understood that the disclosure is not necessarily limited to the specific features or acts described. Rather, the specific features and acts are disclosed as illustrative forms of implementing the embodiments. Conditional language, such as, among others, “can,” “could,” “might,” or “may,” unless specifically stated otherwise, or otherwise understood within the context as used, is generally intended to convey that certain embodiments could include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

### Example Embodiments

Embodiment 1. A method comprising: determining, by one or more computer processors coupled to memory, data associated with a patient, the data comprising a heart rate value, a peak velocity through aortic valve value, and a stroke volume value; generating a flow waveform for the patient using the data; determining a set of candidate vascular impedance values for the patient based at least in part on the flow waveform, the set of candidate vascular impedance values comprising a first vascular impedance value and a second vascular impedance value; determining a first pressure waveform using the flow waveform and the first vascular impedance value; determining a second pressure waveform using the flow waveform and the second vascular impedance value; determining a blood pressure value for the patient; determining that the first pressure waveform is a closer match to the blood pressure value than the second pressure waveform; and determining that the patient has a vascular impedance of the first vascular impedance value.

Embodiment 2. The method of embodiment 1, wherein the data further comprises an aortic valve area value.

Embodiment 3. The method of embodiment 1 or 2, wherein the data is echocardiographic data.

Embodiment 4. The method of embodiment 1 or 2, wherein the data is MRI data.

Embodiment 5. The method of any one of embodiments 1 to 4, further comprising: filtering out the second pressure waveform based at least in part on one or more domain bounding criteria.

Embodiment 6. The method of embodiment 5, wherein the one or more domain bounding criteria comprise pressure waveform upstroke (positive value), ejection duration range, and diastolic decay.

Embodiment 7. The method of any one of embodiments 1 to 6, further comprising: generating a recommendation for an aortic valve replacement procedure for the patient based at least in part on the vascular impedance.

Embodiment 8. The method of any one of embodiments 1 to 6, further comprising: generating a predicted outcome of an aortic valve replacement procedure for the patient based at least in part on the vascular impedance.

Embodiment 9. The method of embodiment 8, wherein generating the predicted outcome comprises selecting the predicted outcome from a predefined set of outcomes.

Embodiment 10. A method for assessing a patient potentially in need of an aortic valve replacement procedure, the method comprising: determining, by one or more computer processors coupled to memory, echocardiographic data associated with the patient, the echocardiographic data comprising a heart rate value, a peak velocity through aortic valve value, an aortic valve area value, and a stroke volume value; generating a flow waveform for the patient using the echocardiographic data; determining a set of candidate vascular impedance values for the patient based at least in part on the flow waveform, the set of candidate vascular impedance values comprising a first vascular impedance value and a second vascular impedance value; determining a first pressure waveform using the flow waveform and the first vascular impedance value; determining a second pressure waveform using the flow waveform and the second vascular impedance value; determining a blood pressure value for the patient; determining that the first pressure waveform is a closer match to the blood pressure value than the second pressure waveform; determining that the patient has a vascular impedance of the first vascular impedance value, and then using the vascular impedance to determine whether the patient is a suitable candidate for an aortic valve replacement procedure.

Embodiment 11. The method of any one of embodiments 1 to 10, wherein determining the first pressure waveform comprises determining the first pressure waveform based at least in part on a Fourier Transform of the flow waveform and the first vascular impedance value.

Embodiment 12. A device comprising: memory configured to store computer-executable instructions; and at least one computer processor configured to access the memory and execute the computer-executable instructions to: determine data associated with a patient, the data comprising a heart rate value, a peak velocity through aortic valve value, and a stroke volume value; generate a flow waveform for the patient using the data; determine a set of candidate vascular impedance values for the patient based at least in part on the flow waveform, the set of candidate vascular impedance values comprising a first vascular impedance value and a second vascular impedance value; determine a blood pressure value for the patient; determine that the first pressure waveform is a closer match to the blood pressure value than the second pressure waveform; and determine that the patient has a vascular impedance of the first vascular impedance value.

Embodiment 13. The device of embodiment 12, wherein the data further comprises an aortic valve area value.

Embodiment 14. The device of embodiments 12 or 13, wherein the data is echocardiographic data.

Embodiment 15. The device of embodiments 12 or 13, wherein the data is MRI data.

Embodiment 16. The device of any one of embodiments 12 to 15, wherein the at least one computer processor is further configured to access the memory and execute the computer-executable instructions to: filter out the second pressure waveform based at least in part on one or more domain bounding criteria.

Embodiment 17. The device of embodiment 16, wherein the one or more domain bounding criteria comprise pressure waveform upstroke (positive value), ejection duration range, and diastolic decay.

Embodiment 18. The device of any one of embodiments 12 to 17, wherein the at least one computer processor is further configured to access the memory and execute the computer-executable instructions to: generate (i) a recommendation for an aortic valve replacement procedure for the patient, and/or (ii) a predicted outcome of an aortic valve replacement procedure for the patient, based at least in part on the vascular impedance.

Embodiment 19. The device of any one of embodiments 12 to 18, wherein the at least one computer processor is configured to access the memory and execute the computer-executable instructions to: determine the first pressure waveform based at least in part on a Fourier Transform of the flow waveform and the first vascular impedance value.

Embodiment 20. A system configured to determine the vascular impedance of a patient according to the method of any one of embodiments 1 to 10.

Modifications and variations of the methods and systems described herein will be obvious to those skilled in the art from the foregoing detailed description. Such modifications and variations are intended to come within the scope of the appended claims.

1. A method comprising:
determining, by one or more computer processors coupled to memory, data associated with a patient, the data comprising a heart rate value, a peak velocity through aortic valve value, and a stroke volume value;
generating a flow waveform for the patient using the data;
determining a set of candidate vascular impedance values for the patient based at least in part on the flow waveform, the set of candidate vascular impedance values comprising a first vascular impedance value and a second vascular impedance value;
determining a first pressure waveform using the flow waveform and the first vascular impedance value;
determining a second pressure waveform using the flow waveform and the second vascular impedance value;
determining a blood pressure value for the patient;
determining that the first pressure waveform is a closer match to the blood pressure value than the second pressure waveform; and
determining that the patient has a vascular impedance of the first vascular impedance value.

2. The method of claim 1, further comprising:
filtering out the second pressure waveform based at least in part on one or more domain bounding criteria.

3. The method of claim 2, wherein the one or more domain bounding criteria comprise pressure waveform upstroke (positive value), ejection duration range, and diastolic decay.

4. The method of claim 1, further comprising:
generating a recommendation for an aortic valve replacement procedure for the patient based at least in part on the vascular impedance.

5. The method of claim 1, further comprising:
generating a predicted outcome of an aortic valve replacement procedure for the patient based at least in part on the vascular impedance.

6. The method of claim 5, wherein generating the predicted outcome comprises selecting the predicted outcome from a predefined set of outcomes.

7. The method of claim 1, wherein determining the first pressure waveform comprises determining the first pressure waveform based at least in part on a Fourier Transform of the flow waveform and the first vascular impedance value.

8. A method for assessing a patient potentially in need of an aortic valve replacement procedure, the method comprising:
determining, by one or more computer processors coupled to memory, echocardiographic data associated with the patient, the echocardiographic data comprising a heart rate value, a peak velocity through aortic valve value, an aortic valve area value, and a stroke volume value;
generating a flow waveform for the patient using the echocardiographic data;
determining a set of candidate vascular impedance values for the patient based at least in part on the flow waveform, the set of candidate vascular impedance values comprising a first vascular impedance value and a second vascular impedance value;
determining a first pressure waveform using the flow waveform and the first vascular impedance value;
determining a second pressure waveform using the flow waveform and the second vascular impedance value;
determining a blood pressure value for the patient;
determining that the first pressure waveform is a closer match to the blood pressure value than the second pressure waveform;
determining that the patient has a vascular impedance of the first vascular impedance value, and then using the vascular impedance to determine whether the patient is a suitable candidate for an aortic valve replacement procedure.

9. A device comprising:
memory configured to store computer-executable instructions; and
at least one computer processor configured to access the memory and execute the computer-executable instructions to:
determine data associated with a patient, the data comprising a heart rate value, a peak velocity through aortic valve value, and a stroke volume value;
generate a flow waveform for the patient using the data;
determine a set of candidate vascular impedance values for the patient based at least in part on the flow waveform, the set of candidate vascular impedance values comprising a first vascular impedance value and a second vascular impedance value;
determine a blood pressure value for the patient;
determine that the first pressure waveform is a closer match to the blood pressure value than the second pressure waveform; and
determine that the patient has a vascular impedance of the first vascular impedance value.

10. The device of claim 9, wherein the data further comprises an aortic valve area value.

11. The device of claim 9, wherein the data is echocardiographic data.

12. The device of claim 9, wherein the data is MRI data.

13. The device of claim 9, wherein the at least one computer processor is further configured to access the memory and execute the computer-executable instructions to:
filter out the second pressure waveform based at least in part on one or more domain bounding criteria.

14. The device of claim 13, wherein the one or more domain bounding criteria comprise pressure waveform upstroke (positive value), ejection duration range, and diastolic decay.

15. The device of claim 9, wherein the at least one computer processor is further configured to access the memory and execute the computer-executable instructions to:
generate (i) a recommendation for an aortic valve replacement procedure for the patient, and/or (ii) a predicted outcome of an aortic valve replacement procedure for the patient, based at least in part on the vascular impedance, and/or
determine the first pressure waveform based at least in part on a Fourier Transform of the flow waveform and the first vascular impedance value.

16. The method of claim 8, wherein determining the first pressure waveform comprises determining the first pressure waveform based at least in part on a Fourier Transform of the flow waveform and the first vascular impedance value.

17. The method of claim 1, wherein the data further comprises an aortic valve area value.

18. The method of claim 1, wherein the data is echocardiographic data.

19. The method of claim 1, wherein the data is MRI data.

20. A system configured to determine the vascular impedance of a patient according to the method of claim 1.
